(19) United States
(12) Patent Application Publication
(10) Pub. No.: US 2024/0066916 A1
(43) Pub. Date: Feb. 29, 2024

(54) WHEEL FOR A VEHICLE AND SYSTEM COMPRISING THE WHEEL

(71) Applicants: Wuhan Lotus Cars Co., Ltd, Wuhan, Hubei (CN); NINGBO GEELY AUTOMOBILE RESEARCH & DEVELOPMENT CO., LTD., Zhejiang (CN)

(72) Inventors: Franco Cimatti, Pavullo nel Frignano (IT); Ehab Fares, Zwingenberg (DE)

(21) Appl. No.: 18/259,666

(22) PCT Filed: Dec. 28, 2020
(86) PCT No.: PCT/CN2020/140398

§ 371 Date: Jun. 28, 2023

Related U.S. Application Data
(60) Provisional application No. 62/976,735, filed on Feb. 14, 2020.

(51) Int. Cl. B60B 19/10; B60B 3/10; F16D 65/847

(52) U.S. Cl. CPC B60B 19/10; B60B 3/10; F16D 65/847; B60B 2900/513

(57) ABSTRACT

The invention refers to a wheel for a vehicle, the wheel being rotatable around a rotation axis and comprising: a seating space for a brake disk extending around the rotation axis; a wheel rim having a rim well, the wheel rim extending around the seating space and the rotation axis, wherein the rim well protrudes from the wheel rim towards the rotation axis; and at least one spoke being connected to the wheel rim, the at least one spoke being arranged at an outward side of the wheel and extending at least in part radially to the rotation axis; wherein, along the rotation axis, the seating space is arranged closer to the outward side than the rim well. The invention improves the air flow in the vicinity of the wheel to reduce aerodynamic drag and lifting forces on the vehicle.

### TECHNICAL FIELD

The invention relates to a wheel for a vehicle and system comprising the wheel.

### BACKGROUND ART

Wheels for cars comprise a wheel rim which has an outer radial surface for supporting a tire. The wheels are attached to brakes to reduce the rotation velocity of the wheels and to brake the car. Conventional wheel rims encompass a brake. The brake reduces the speed of the wheel by converting the kinetic energy of the car to thermal energy. This conversion heats up the brake. Thus, the brake requires cooling. The cooling system of the brake uses air for absorbing the thermal energy. The air is guided along and/or through channel on and/or in the brake. A hub for the wheel leads air from the underside of the car towards the brake. That air is used to cool the brake disk. After the air absorbed thermal energy from the brake, the air leaves the channels in the brake. The leaving air is directed towards the wheel rim such that the air strikes the wheel rim. The wheel rim redirects the heated air coming from the brake to the underside of the car. Such an air flow induces aerodynamic drag and lift forces on the car.

DE 43 42 138 A1 describes a wheel rim for a car. The wheel rim comprises a fan for providing an air flow from the outward side of the wheel towards a brake disk being encompassed by the wheel. The air flow streams through channels in the brake disk for cooling the brake disk. After leaving the channels, the wheel rim guides the air flow under the car.

### SUMMARY OF INVENTION

### Technical Problem

There is a need for improving the air flow in the vicinity of the wheel to reduce aerodynamic drag and lifting forces on the car, further.

### Solution to Problem

### Technical Solution

claims 1, 7 and 11 describe features of the invention. Claims 2 to 6 and 8 to 10 describe exemplary embodiments of the invention.

According to a first aspect of the invention, a wheel for a vehicle is provided, the wheel being rotatable around a rotation axis and comprising: a seating space for a brake disk extending around the rotation axis; a wheel rim having a rim well, the wheel rim extending around the seating space and the rotation axis, wherein the rim well protrudes from the wheel rim towards the rotation axis; and at least one spoke being connected to the wheel rim, the at least one spoke being arranged at an outward side of the wheel and extending at least in part radially to the rotation axis; wherein, along the rotation axis, the seating space is arranged closer to the outward side than the rim well.

The invention provides a wheel which directs air coming from the seating space of a brake disk towards the outward side of the wheel. The outward side of the wheel faces away from the car, when the wheel is mounted on the car. The wheel comprises a wheel rim having a rim well that extends around a rotation axis. The wheel is rotatable around the rotation axis. The rim well furthermore protrudes from the wheel rim in radial direction towards the rotation axis. Furthermore, the wheel rim extends around a seating space for a brake disk. The seating space may receive a brake disk when the wheel is mounted on a car. Along the rotation axis the seating space is between the rim well and the outboard side of the wheel. A brake disk mounted in the seating space will blow air out towards the wheel rim. Air coming from the seating space strikes the wheel rim between the rim well and the outward side of the wheel. The air then flows along the wheel rim. Since the rim well protrudes towards the rotation axis, the rim well is an obstacle for air flowing away from the outward side of the wheel. The rim well therefore deflects that air and redirects that air towards the outward side. Thus, most of the air striking the wheel rim flows towards the outward side. This air flow reduces the aerodynamic drag in the vicinity of the wheel by reducing turbulences in the vicinity of the wheel and the wheel house at the vehicle. Furthermore, the air flow induces a pressure reduction at the underside of the car which counters lifting forces. In addition, it is avoided that air being heated by the brake disk recirculates in the wheel house and returns to the brake disk. This improves the cooling effect of the brake disk. Thus, the invention improves the air flow in the vicinity of the wheel, reducing aerodynamic drag and lifting forces on the car.

In an example, the wheel rim may comprise an air guide portion between the rim well and the outward side, wherein the air guide portion may be configured to guide air towards the outward side.

The air guide portion may increase and optimize the air flow from the rim well towards the outward side. Thus, the aerodynamic drag and the lifting forces on the car are reduced, further.

In another example, the air guide portion may be tapered, wherein, along the rotation axis, the air guide portion may comprise a diameter increasing towards the outward side.

Air coming from the seating space may then strike the wheel rim on the tapered air guide portion. Due to the tapered shape, in a direction from the rim well to the outward side, the air guide portion has an angle being bigger than 90° to the radial direction. Thus, the air coming from the seating space striking the tapered air guide portion may be deflected to the outward side. This further increases the air flow towards the outward side and further reduces the aerodynamic drag and the lifting forces on the car.

According to an exemplary embodiment, the at least one spoke may be configured to accelerate air, which streams from the rim well towards the outward side, if the wheel turns in a predefined rotation direction around the rotation axis.

This may increase the air flow towards the outward side further. The increased air flow results in an increase of suction of air from the underside of the car. Thus, the aerodynamic drag is reduced, further.

Furthermore, in an example, the at least one spoke may comprise a blade portion extending between the outward side and the rim well, wherein the blade portion may be configured to accelerate air towards the outward side, if the wheel turns in the predefined rotation direction around the rotation axis.

This may increase the air flow towards the outwards side with a simple means.

In a further example, along the rotation axis, the at least one spoke may have an asymmetrical shape.

Due to the asymmetrical shape, the spoke may redirect air flowing out through the outwards side away from the axial direction. Furthermore, the asymmetrical shape may accelerate the air flow through the outward side, further.

According to another aspect of the invention, a system is provided, the system comprising a brake disk, a wheel hub connectable to a vehicle, and a wheel according to above description, wherein the brake disk is arranged in the seating space, wherein the brake disk and the wheel are attached to the wheel hub.

The effects and further embodiments of the system according to the present invention are analogous to the effects and embodiments of the wheel according to the description mentioned above. Thus, it is referred to the above description of the wheel.

According to an example, the brake disk may comprise at least one cooling air channel extending at least in part radially to the rotation axis, wherein the cooling air channel may be configured to thrust out air towards the wheel rim.

In another example, the cooling air channel comprises an outlet opening facing the wheel rim between the rim well and the outward side.

The air thrusted out towards the wheel rim may strike the wheel rim between the rim well and the outward side. Due to the protrusion of the rim well towards the rotation axis most of the air may flow towards the outwards side.

Furthermore, in a further example, the wheel hub comprises a guiding channel being configured to guide air from an inward side of the wheel towards the brake disk, the inward side being opposite to the outward side.

In a further aspect, a vehicle is provided, the vehicle comprising a wheel according to above description and/or a system according to the above description, wherein the wheel and/or the system is mounted the vehicle in a rotatable manner.

The effects and further embodiments of the vehicle according to the present invention are analogous to the effects and embodiments of the wheel and/or the system, respectively, according to the description mentioned above. Thus, it is referred to the above description of the wheel and/or the system, respectively.

### Advantageous Effects of Invention

### Advantageous Effects

The results in an air flow from the underside of the vehicle through the wheel without a lot of turbulences in the vicinity of the wheel. Due to the reduced amount of turbulences, the aerodynamic drag improves due to that air flow. Furthermore, the air flow leaving the underside of the vehicle causes a pressure reduction between the vehicle and the ground. This reduces lifting forces, which result from the movement of the vehicle.

### BRIEF DESCRIPTION OF DRAWINGS

### Description of Drawings

The following description of exemplary embodiments, the wording of the claims and the drawings provide further features, details and advantages of the invention. The figures show:

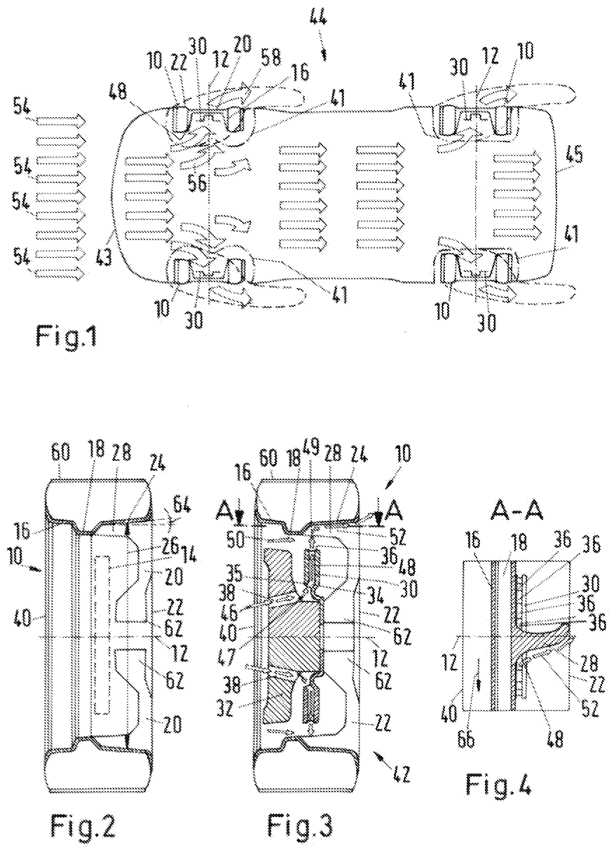

FIG. 1 a schematic drawing of a vehicle from a bottom view;

FIG. 2 a schematic radial cross section of a wheel;

FIG. 3 a schematic radial cross section of a system with a wheel; and

FIG. 4 a schematic cross section of a system according to line A-A of FIG. 3.

### BEST MODE FOR CARRYING OUT THE INVENTION

### Best Mode

FIG. 1 shows a vehicle 44 from below. The vehicle 44 of this example is a car having four wheels 10. The wheels 10 are arranged in wheel houses 41 of the vehicle 44.

Each wheel 10 is rotatable around a rotation axis 12 and comprises an outward side 22 facing away from the car and an inward side 44 facing the car. Each wheel 10 is attached to a brake disk 30. The brake disk 30 is arranged between the wheel 10 and the car 44, i.e. the brake disk 30 is arranged on the inward side 40 of the wheel 10.

During movement of the vehicle 44, air indicated by arrows 54 flows under the vehicle 44 from the nose 43 of the vehicle 44. That air further flows along the underside of the vehicle 44 and leaves the vehicle 44 on the tail 45 of the vehicle 44.

In the vicinity of the wheel houses 41, a portion of the air indicated by arrows 56 is flowing to the wheel house 41 towards the wheels 10.

FIG. 2 shows a detailed schematic cross section of the wheel 10 in a direction radial to the rotation axis 12.

The wheel 10 may comprise a wheel rim 16, a tire 60, and at least one spoke 20. The tire 60 is mounted on a radial outward facing side of the wheel rim 16.

The wheel rim 16 comprises a rim well 18, which protrudes in radial direction towards the rotation axis 12. Furthermore, the wheel rim 16 extends around the rotational axis 12 and encompasses a seating space 14 for a brake disk 30. The seating space 14 is configured to receive a brake disk 30, when the wheel 10 is mounted on a vehicle 44.

Along a direction being parallel to the rotation axis 12, the seating space 14 is closer to the outward side 22 than the rim well 18. The rim well 18 is arranged closer to the inward side 40 than the seating space 14. Thus, the rim well 18 is arranged inboard of the wheel 10.

The wheel rim 16 further comprises an air guide portion 24 extending between the rim well 18 and the outward side 22. The air guide portion 24 is configured to guide air coming from the seating space 14 towards the outward side 22. Furthermore, the air guide portion 24 comprises a tapered shape, wherein a diameter of the wheel rim 16 at the air guide portion 24 increases from the rim well 18 towards the outward side 22.

The at least one spoke 20 is arranged on the outward side 22 of the wheel 10. The spoke 20 extends from the wheel rim 16 towards the rotation axis 12. Furthermore, the spoke 20 comprises an attachment portion 62 close to the rotation axis 12. The attachment portion 62 is configured to be attached to a vehicle 44. For example, the attachment portion 62 may comprise at least one opening for inserting an attachment means like a screw, a bolt or a rivet.

The spoke 22 may further comprise a blade portion 28 extending parallel to the rotational axis along at least a portion of the air guide portion 24. The blade portion 28 may attach the at least one spoke 22 to the wheel rim 16.

FIG. 3 shows a schematic cross section of a system 42 comprising the wheel 10, a brake disk 30 and a wheel hub 32. The wheel 10 and the brake disk 30 are attached to the wheel hub 32.

The wheel hub 32 may be attached to the vehicle 44. Furthermore, the wheel hub 32 comprises at least one guiding channel 38 that is configured to guide air from the underside of the vehicle 44 towards the brake disk 30. Arrow 46 indicates the air coming from the underside of the vehicle 44 and being received and guided by the guiding channel 38.

The brake disk 30 is arranged in the seating space 14 of the wheel 10 and comprises at least one cooling air channel 34. In this example, the brake disk 30 comprises a plurality of cooling air channels 34. The cooling air channel 34 may extend inside the brake disk 30 or on a surface thereof facing the wheel hub 32 between an inlet opening 35 and an outlet opening 36. The inlet opening 35 receives the guided air being indicated by arrow 47 from the guiding channel 38. Arrow 48 indicates the air flowing through the cooling air channel 34 that cools the brake disk 30.

When the brake disk 30 rotates the air in the cooling air channel 34 is accelerated by centrifugal forces towards the outlet opening 36. The air that cools the brake disk 30 in the cooling air channel 34 leaves the cooling air channel 34 through the outlet opening 36. The outlet opening 36 is arranged such that air flowing out of the outlet opening 36 strikes the wheel rim 16 at the air guide portion 24 between the rim well 18 and the outward side 22. The air striking the air guide portion 24 is indicated by arrow 49. In this example, that air has a flow component that is radial to the rotation axis 12.

When striking the air guide portion 24, the air is deflected towards the outward side 22. The arrow 52 indicates that air flow. The tapered shape of the air guide portion 24 supports that deflection since in the direction towards the outboard side 22, the tapered shape has an angle 64 with the radial direction that is bigger than 90°. The angle 64 may be in the range between 90° and 100°. In particular, the angle 64 may be 95°.

Furthermore, the rim well 18 protruding towards the rotational axis 12 also causes air 49 coming from the seating space 14 to flow towards the outward side 22. The rim well 18 is an obstacle for air that would flow towards the inward side 40. Therefore, the rim well 18 deflects the air striking the air guide portion 24 close to the rim well 18 towards the outward side 22.

The deflected air flowing along the air guide portion 24 induces a suction on the air being arranged at the inward side 40 of the wheel 10. That air is therefore drawn towards the outward side 22. Arrow 50 indicates that air. Thus, additional air from the underside of the vehicle 44 flows from through the inward side 40, the wheel 10 to the outward side 22.

The blade portion 28 extending along the air guide portion 24 is configured to accelerate the air flowing along the air guide portion 24 towards the outward side 22. This is shown in detail in in FIG. 4 that is a cross section along line A-A in FIG. 3 in a view towards the rotation axis 12.

FIG. 4 indicates the preferred rotation direction of the wheel 10 with arrow 66. The arrow 66 indicates that the shown portion of the rim well 18 and the wheel rim 16 move in the direction of the arrow 66, when the wheel 10 rotates around the rotation axis 12. When mounting the wheel 10 on a vehicle 44 such that in the forward movement direction of the vehicle 44, the wheel 10 rotates in the preferred rotation direction, the preferred rotation direction is the predefined rotation direction.

According to FIG. 4, the blade portion 28 comprises an asymmetric shape. At the outward side 22, a portion of the blade portion 28 is bend away from the predefined rotation direction. Thus, the air 52 flowing along the air guide portion 24 is deflected away from the rotation direction. This causes a suction force, which accelerates the air flowing along the air guide portion 24 towards the outward side 22.

In an alternative exemplary embodiment of the at least one spoke 20, the blade portion 28 comprises a symmetric shape. In that exemplary embodiment, the blade portion 28 may also accelerate the air flowing along the air guide portion 24. The accelerating effect is weaker than in the example of the asymmetric blade portion 28. However, the accelerating effect occurs in both rotation directions. Thus, the acceleration effect of the symmetric blade portion 28 is independent of the rotation direction of the wheel 10.

In total, air streams from the inward side 40 through the wheel 10 to the outward side 22. In a first path through the wheel 10, the air may flow through the wheel hub 32 and the brake disk 30. In a further path, the air flows from the inward side 40 around the wheel hub 32, along the wheel rim 16 towards the outward side 22.

This results in an air flow from the underside of the vehicle 44 through the wheel 10 without a lot of turbulences in the vicinity of the wheel 10. Due to the reduced amount of turbulences, the aerodynamic drag improves due to that air flow. Furthermore, the air flow leaving the underside of the vehicle 44 causes a pressure reduction between the vehicle 44 and the ground. This reduces lifting forces, which result from the movement of the vehicle 44.

The invention is not limited to one of the described embodiments, but can be modified in many ways. All features and advantages resulting from the claims, the description and the drawing, including constructional details, spatial arrangements and procedural steps, may be essential to the invention, both individually and in various combinations.

### LIST OF REFERENCE SIGNS

10:wheel; 12:rotation axis; 14:seating space; 16:wheel rim; 18:rim well; 20:spoke; 22:outward side; 24:air guide portion; 26:diameter; 28:blade portion; 30:brake disk; 32:wheel hub; 34:cooling air channel; 35:inlet opening; 36:outlet opening; 38:guiding channel; 40:inward side; 41:wheel house; 42:system; 43:nose; 44:vehicle; 45:tail; 46:air flow; 47:air flow; 48:air flow; 50:air flow; 52:air flow; 54:air flow; 56:air flow; 58:air flow; 60:tire; 62:attachment portion; 64:angle; 66:rotation direction.

1. A wheel for a vehicle, the wheel being rotatable around a rotation axis and comprising:
a seating space for a brake disk extending around the rotation axis;
a wheel rim having a rim well, the wheel rim extending around the seating space and the rotation axis, wherein the rim well protrudes from the wheel rim towards the rotation axis; and
at least one spoke being connected to the wheel rim, the at least one spoke being arranged at an outward side of the wheel and extending at least in part radially to the rotation axis;
wherein, along the rotation axis, the seating space is arranged closer to the outward side than the rim well.

2. The wheel according to claim 1, wherein the wheel rim comprises an air guide portion between the rim well and the outward side, wherein the air guide portion is configured to guide air towards the outward side.

3. The wheel according to claim 2, wherein the air guide portion is tapered, wherein, along the rotation axis, the air guide portion comprises a diameter increasing towards the outward side.

4. The wheel according to claim 1, wherein the at least one spoke is configured to accelerate air, which streams from the rim well towards the outward side, if the wheel turns in a predefined rotation direction around the rotation axis.

5. The wheel according to claim 4, wherein the at least one spoke comprises a blade portion extending between the outward side and the rim well, wherein the blade portion is configured to accelerate air towards the outward side, if the wheel turns in the predefined rotation direction around the rotation axis.

6. The wheel according to claim 4, wherein, along the rotation axis, the at least one spoke has an asymmetrical shape.

7. A system comprising the wheel according to claim 1, the brake disk, and a wheel hub connectable to a vehicle, wherein the brake disk is arranged in the seating space, wherein the brake disk and the wheel are attached to the wheel hub.

8. The system according to claim 7, wherein the brake disk comprises at least one cooling air channel extending at least in part radially to the rotation axis, the cooling air channel being configured to thrust out air towards the wheel rim.

9. The system according to claim 8, wherein the cooling air channel comprises an outlet opening facing the wheel rim between the rim well and the outward side.

10. The system according to claim 7, wherein the wheel hub comprises a guiding channel being configured to guide air from an inward side of the wheel towards the brake disk, the inward side being opposite to the outward side.

11. A vehicle comprising the wheel according to claim 1, wherein the wheel is mounted the vehicle in a rotatable manner.
